(19) United States
(12) Patent Application Publication
(10) Pub. No.: US 2024/0293061 A1
(43) Pub. Date: Sep. 5, 2024

(54) URINE SAMPLE COLLECTION DEVICE AND METHODS OF USE

(71) Applicant: FidaLab, LLC, Seattle, WA (US)

(72) Inventors: Qinghua Feng, Seattle, WA (US); Mujun Yu, Seattle, WA (US); Paul D. Seiter, San Diego, CA (US)

(73) Assignee: FidaLab, LLC, Seattle, WA (US)

(21) Appl. No.: 18/177,743

(22) Filed: Mar. 2, 2023

(51) Int. Cl. A61B 5/20; A61F 5/451

(52) U.S. Cl. CPC A61B 5/207; A61F 5/451

(57) ABSTRACT

A sample collection device includes an inlet, a waste chamber having a waste chamber opening, a collection chamber having a collection chamber opening, and a toggling mechanism configured to select between a first fluid path from the inlet to the waste chamber via the waste chamber opening, and a second fluid path from the inlet to the collection chamber via the collection chamber opening.

### FIELD OF THE DISCLOSURE

The present disclosure relates to a specimen collection device and, more particularly, a collection device having a mechanism that separates the user's initial discharge from the desired sample.

### BACKGROUND OF THE DISCLOSURE

Urine samples are collected by discarding the first portion of the urine stream and only collecting the second portion for microbiological testing. For example, the periurethral skin may be contaminated with bacteria which leads to false test results including a false positive for urinary tract infection, which, in turn, may lead to unnecessary treatment and/or medication and inappropriate patient management. To prevent misdiagnosing the patient, the user may be instructed to only capture the “mid-stream” portion of a sample and to discard the initial stream.

Complications may occur when obtaining the sample with patients who have difficulty catching the urine “mid-stream”. This includes, but is not limited to, small children who are not potty trained, incontinent adults, and frail individuals.

Additionally, sample collection (e.g., urine sample collection) may be time-consuming, especially when samples are needed from multiple patients or users. These delays may limit the total testing volume within a hospital, clinic, care facility, or sample collection site.

### SUMMARY OF THE DISCLOSURE

In some examples, a sample collection device includes an inlet, a waste chamber having a waste chamber opening, a collection chamber having a collection chamber opening, and a toggling mechanism configured to select between a first fluid path from the inlet to the waste chamber via the waste chamber opening, and a second fluid path from the inlet to the collection chamber via the collection chamber opening.

### BRIEF DESCRIPTION OF THE DISCLOSURE

Embodiments of the presently disclosed urine sample collection devices and systems are shown herein with reference to the drawings, wherein:

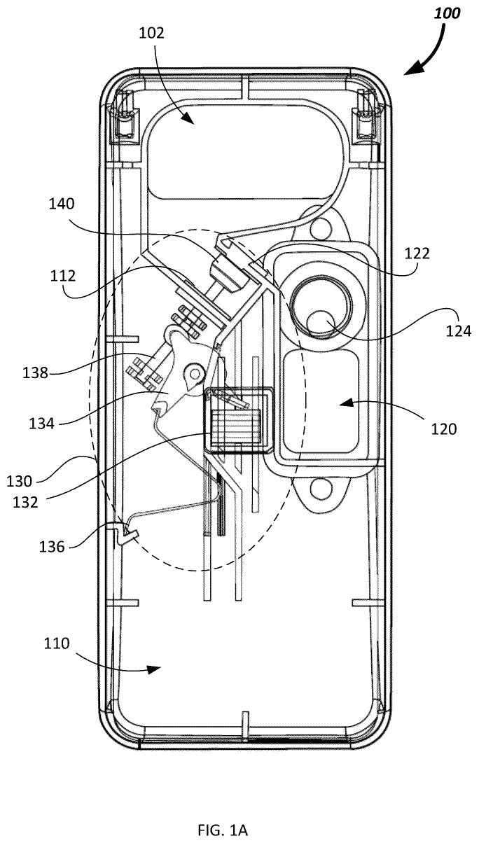

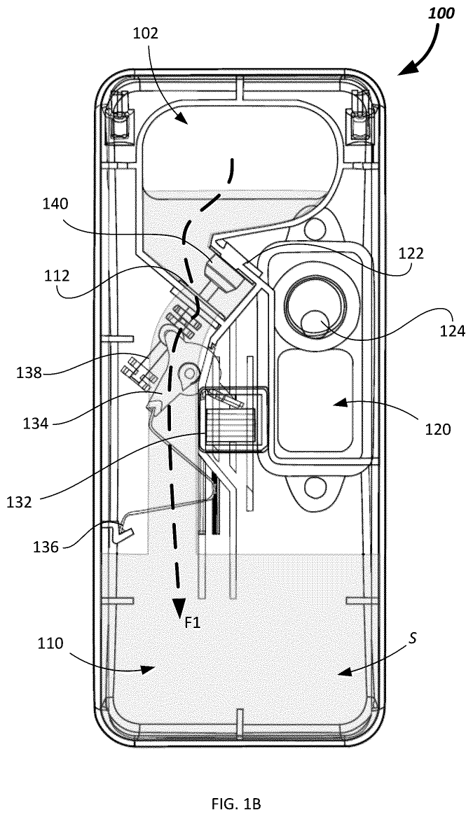

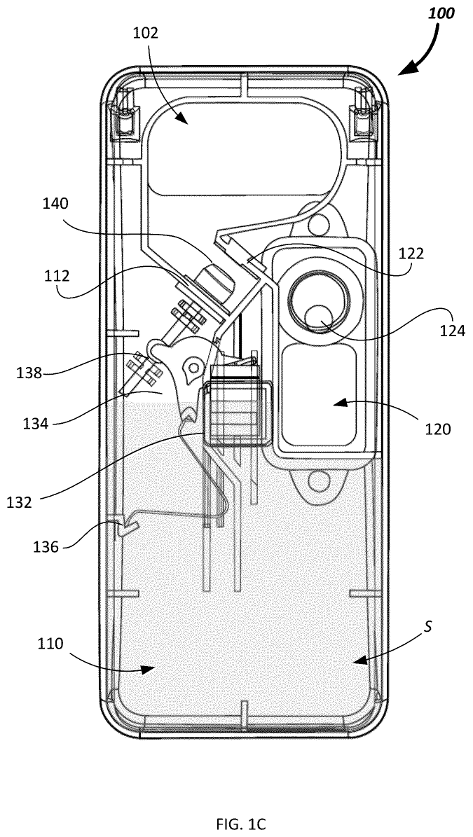

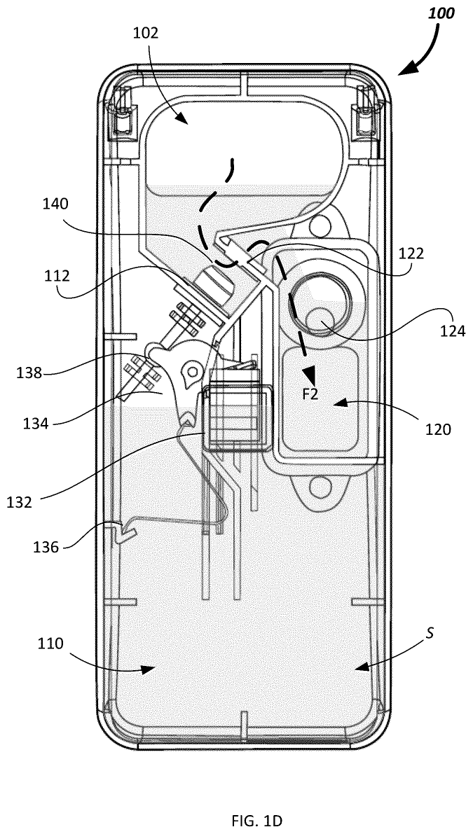

FIGS. 1A-D are schematic cross-sectional views of a urine sample collection device at various stages of use; and

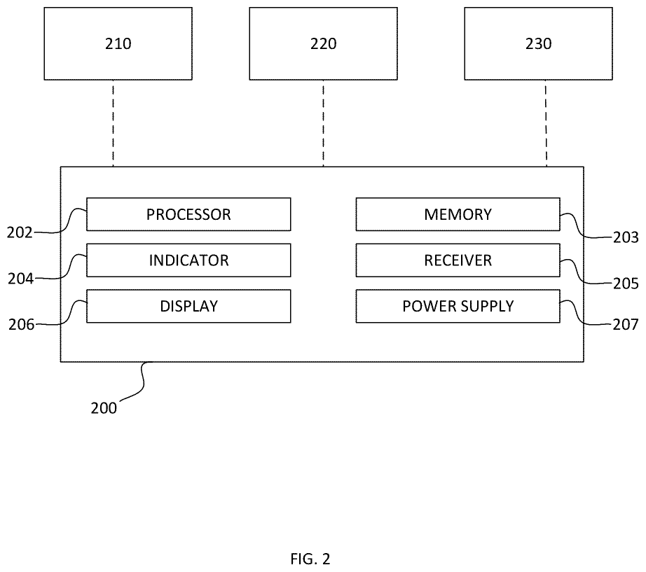

FIG. 2 is a schematic block diagram of a base unit capable of communicating with multiple sample collection devices.

The embodiment of the present invention will now be described with reference to the appended drawings. It is to be appreciated that these drawings depict only some embodiments of the invention and are therefore not to be considered limiting of its scope.

### DETAILED DESCRIPTION

Despite the various improvements that have been made to urine sample collection devices and their methods of use, conventional devices suffer from some shortcomings as described above.

There therefore is a need for further improvements to the devices, systems, and methods of collecting and sorting collected specimen. Among other advantages, the present disclosure may address one or more of these needs.

FIG. 1A illustrates one example of a urine sample collection device 100. The sample collection device 100 generally includes an inlet 102 where the patient's specimen is received, two chambers 110,120 and a toggling mechanism 130 for selecting one of the chambers. Generally, the initially received specimen is directed from the inlet 102 into a waste chamber 110, which has a waste chamber opening 112 for the specimen to enter. A toggling mechanism 130 allows the continuous stream of specimen to be directed toward either waste chamber 110 or a collection chamber 120 via the collection chamber opening 122.

In one example, toggling mechanism 130 may comprise a spring 136 in contact with a wall of the device 100 at a first end, and with a lever 134 at a second end. The spring 136 may bias lever 134 into an initial condition, which in turn actuates an arm 138 connected to a plug 140 to seal the collection chamber opening 122. The lever 134 may also have a second condition in which the arm 138 is actuated to seal the waste chamber opening 122. Adjacent, or within, waste chamber 110, a sponge 132 is disposed which will absorb a portion of the sample as the waste chamber fills. Absorption of fluid by the sponge 132 may also actuate lever 134 as will be described below in greater detail.

FIG. 1B illustrates the specimen “S” being directed through inlet 102 and flowing into waste chamber 110 through the waste chamber opening 112, through first fluid path F1. As specimen “S” continues to fill up waste chamber 110, it will reach a position where it fills the waste chamber 110 and contact sponge 132. The sponge 132, in turn, will absorb a portion of specimen “S” and enlarge, as seen in FIG. 1C. This enlargement may include axial elongation, which forces the sponge 132 to contact and/or actuate lever 134. In this example, lever 134 will rotate into a second condition, compressing the spring 136 and allowing the arm 138 to move diagonally away from collection chamber opening 122, and to cover the waste chamber opening 112, sealing or closing the waste chamber 110.

As seen in FIG. 1D, further flow of specimen “S” may be collected through the inlet 102 after the plug 140 has fully blocked the waste chamber opening 112. With the waste chamber 110 properly sealed, the specimen will begin to fill the collection chamber 120 through the collection chamber opening 122 via second fluid path F2. In this manner, the toggling mechanism 130 will automatically capture a portion of the sample “midstream” in the collection chamber 120 without action by the user. Notably, the toggling mechanism 130 is actuated with the moisture absorption of the sponge and without user interference.

In one example, sample collection device 100 may include a battery, a telemetry unit (e.g., BLUETOOTH®, WiFi, etc.), and an optional moisture sensor 124 disposed within the collection chamber 120 at a predetermined height. As the specimen “S” fills collection chamber 120, it may reach the moisture sensor 124, which detects and/or confirms the presence of a sample within the collection chamber 120. A message from the sample collection device 100 may be sent to other device(s) via the telemetry unit to alert the other devices as to a status of the collection chamber 120.

In some embodiments, one or more sample collection device(s) may be coupled to a base unit. FIG. 2 shows one such example, in which a base unit device 200 receives signals from three sample collection devices 210,220,230. The sample collection devices 210,220,230 may each collect a sample from a different patient, and each may include a unique moisture sensor 124. The base unit device 200 may serve as a central hub for collecting and/or processing data from the sample collection devices 210,220,230, and is designed to provide a reliable and convenient way for healthcare clinicians to monitor the completion of sample collection. Although three sample collection devices are shown, it will be understood that a base unit device 200 may be paired to a single sample collection device, two, three, four, five, six, seven, eight or more devices.

The base unit device 200 may comprise several components, including a processor 202, a memory 203, an indicator 204, a receiver 205 (e.g., BLUETOOTH®, WiFi, etc.), a display 206 (e.g., LCD) and a power supply 207 (e.g., a battery or power outlet). The processor 202 may be responsible for receiving the incoming signals via the receiver 205 or wireless communication module from one or more of the sample collection devices 210,220,230 and storing the data in the memory 203. Additionally, the base unit device 200 may trigger the indicator 204 to alert the clinician when a sample collection device 210,220,230 has detected moisture within a corresponding collection chamber 120.

In one example, when the base unit device 200 receives a signal indicating the presence of moisture from a sample in one of the sample collection devices, it may activate the indicator 204 to notify the clinician. In some examples, the indicator 204 may be one or more LED lights, an audible alert, a tactile alert, a text message sent to another device (e.g., a clinician's cellular phone or tablet), a message on display 206, etc. The indicator 204 may provide an efficient and convenient way for the clinician to monitor the moisture content of multiple devices simultaneously, and to collect the sample when it is ready.

In use, samples may be taken from multiple patients or users, and each user may have a designated sample collection device. The sample collection devices will separate each sample (e.g., urine) into two chambers. For example, an initial stream or sample may be sent to a waste collection chamber, and a second or subsequent stream or sample may be sent to the sample collection chamber. A toggling mechanism in each of the sample collection devices may automatically separate the sample into the two chambers. Additionally, when the sample collection chamber is filled or has a sufficient volume, then a moisture sensor may detect the presence of the sample and send a signal to a base unit. The base unit, in turn, may communicate with and/or monitor several sample collection devices and alert the user that one or more of the samples are ready for collection. In this manner, a single clinician may oversee or manage the collection of samples from multiple patients or users at the same time.

In some examples, the base unit may form part of a system for logging and storing information related to sample collection and patient information. Information acquired from a sample collection device may be logged and stored in the memory 203 of a base unit. This information may include any of the following data, but is not limited to, the patient or user's name, date of birth, gender, details of the assigned sample collection devices 210,220,230 (e.g., location, serial number, etc.), the date and/or time of the sample collection, whether the user was fasting, the purpose of the sample collection, the status of the sample collection, the volume of the sample and other relevant parameters and/or results. Before the testing process begins, the user's information may be collected and sent to the base unit 200 and stored in the memory 203. Upon completion of the sample collection, the sample collection device 100 may inform the base unit 200 of the detection of moisture within the device and update the completion status. The results may be recorded on the base unit 200 and saved in the memory 203 for access by a clinician at any time.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

What is claimed is:

1. A sample collection device, comprising:
an inlet;
a waste chamber having a waste chamber opening;
a collection chamber having a collection chamber opening; and
a toggling mechanism configured to select between a first fluid path from the inlet to the waste chamber via the waste chamber opening, and a second fluid path from the inlet to the collection chamber via the collection chamber opening.

2. The sample collection device of claim 1, wherein the toggling mechanism comprises a sponge disposed adjacent the waste chamber.

3. The sample collection device of claim 2, further comprising a lever configured and arranged to be actuated by the sponge, the lever being coupled to an arm terminating in a plug.

4. The sample collection device of claim 3, further comprising a spring configured to bias the lever into an initial condition that opens the first fluid path.

5. The sample collection device of claim 4, wherein absorption of moisture by the sponge actuates the lever into a second condition to open the second fluid path.

6. The sample collection device of claim 3, wherein the plug is configured to seal only one of the waste chamber opening and the collection chamber opening at a time.

7. A system for collecting a sample, comprising:
one or more sample collection devices, each of the one or more sample collection devices having a designated moisture sensor; and
a base unit configured and arranged to receive a signal from the one or more sample collection devices, and alert a user when moisture is detected by the designated moisture sensor.

8. The system of claim 7, wherein the base unit comprises a processor and a memory.

9. The system of claim 7, wherein the base unit comprises an indicator for alerting the user to a presence of moisture within at least one of the one or more sample collection devices.

10. The system of claim 8, wherein the memory is configured and arranged to store and log information from the one or more sample collection devices.

11. The system of claim 9, wherein the indicator comprises an audible alert.

12. The system of claim 9, wherein the indicator comprises a message on a display.

13. The system of claim 9, wherein the indicator comprises one or more lights.

14. A method of collecting a sample, comprising:
providing one or more sample collection devices, each of the one or more sample collection devices having a designated moisture sensor;
sending a signal from the one or more sample collection devices to a base unit; and
alerting a user via the base unit when moisture is detected by the designated moisture sensor.

15. The method of claim 13, wherein alerting the user comprises producing an audible alert.

16. The method of claim 13, wherein alerting the user comprises sending a message to a mobile device.

17. The method of claim 13, wherein alerting the user comprises powering an LED on the base unit.

18. The method of claim 13, wherein the one or more sample collection devices comprises a plurality of sample collection devices.

19. The method of claim 14, further comprising logging information from the one or more sample collection devices.

20. The method of claim 19, wherein the information includes a date and time of the sample collection.
